(19) United States
(12) Patent Application Publication
(10) Pub. No.: US 2026/0241187 A1
(43) Pub. Date: Aug. 20, 2026

(54) METHOD FOR CONTROLLING NUMBER OF ADIPOCYTES

(71) Applicants: SUMIDA CORPORATION, Tokyo (JP); TOYO UNIVERSITY, Tokyo (JP)

(72) Inventors: Masaki SAITO, Miyagi (JP); Masafumi OHSAKO, Tokyo (JP); Hirai SUITO, Tokyo (JP)

(21) Appl. No.: 19/473,369

(22) PCT Filed: Feb. 20, 2024
(86) PCT No.: PCT/JP2024/006001

§ 371 Date: Oct. 7, 2025

(30) Foreign Application Priority Data
Apr. 14, 2023 (JP) 2023-066163

(51) Int. Cl. A61N 1/40

(52) U.S. Cl. CPC A61N 1/40

(57) ABSTRACT

A method of controlling the number of adipocytes in a tissue of a living body is provided. The method of controlling the number of adipocytes includes imparting electrical stimulation to the tissue of the living body using a vector potential generator, in which the electrical stimulation can increase or reduce the number of adipocytes by controlling the frequency of an alternating current applied to the vector potential generator.

### CROSS REFERENCE TO REPLATED APPLICATIONS

This application is a 371 U.S. National Phase of International Application No. PCT/JP2024/006001, filed on Feb. 20, 2024, which claims priority to Japanese Patent Application No. 2023-066163, filed on Apr. 14, 2023. The entire disclosures of the above applications are incorporated herein by reference.

### BACKGROUND

### Technical Field

The present invention relates to a method for controlling the number of adipocytes in a tissue of a living body using a vector potential generator, and the like.

### Related Art

Conventionally, to reduce fat, reduction of fat itself is known to occur when cells need energy due to external motion stimuli and the like and fat is reduced when used and converted into energy. In addition, therapy using stimulation by energization is also used for slimming (dieting), either in clinical practice or in daily life. For example, Patent Literature 1 discloses a biomedical-electrode fitting 1 to be worn on a part of a body and describes that the fitting is to be used for “electrical muscle stimulation (EMS) that causes a muscle 50 to expand and contract by applying a training stimulus signal that is an electric stimulus signal to a body surface of the muscle 50” and that “the purpose of training the muscle 50 may be any of dieting by expanding and contracting the muscle 50 to burn fat in a vicinity, improving muscle strength, performing regenerative medicine to regenerate the muscle 50 having been damaged, and the like and the biomedical-electrode fitting 1 may be used for any of these purposes” (refer to paragraph 0045 in Patent Literature 1).

However, these therapies require closely attaching electrodes to a body surface of at least a part of the body at a prescribed contact pressure. To provide more effective electrical stimulation, a patient is to be subjected to an extremely shocking stimulation such as performing surgery on the patient or inserting acupuncture needles through the skin to an affected area. In addition, such shocking stimulation may cause the patient's skin and muscle to contract and end up reducing effectiveness of treatment.

On the other hand, a non-contact spatial electric field generator that can generate a linear electric field and work externally by generating a vector potential without generating a magnetic field is disclosed (for example, refer to Patent Literature 2). In addition, it has been reported that electrical stimulators based on this principle, which have shorter healing times, are easier on the body, and are easier to install, can treat fractures, osteoporosis, and other injuries to the human body, as well as tumors (for example, refer to Patent Literature 3).

### CITATION LIST

### Patent Literature

Patent Literature 1: Japanese Patent Laid-Open No. 2018-114093
Patent Literature 2: International Publication No. WO 2015/099147
Patent Literature 3: Japanese Patent No. 7151356

### Technical Problem

However, a significant amount of exercise is required to reduce fat through exercise therapy and, given that the method of exercise varies from one individual to the next, effectiveness of fat reduction also varies. In addition, with therapy using stimulation by energization, there is also damage to the skin caused by the fitting and wearing the fitting for a long period of time itself may conceivably be quite painful.

In consideration thereof, an object of the present disclosure is to clarify what kind of effect electrical stimulation by a vector potential generator such as that disclosed in Patent Literature 3 has on adipocytes and to provide a method for controlling the number of adipocytes using the apparatus.

### SUMMARY

The present disclosure has been made in order to solve the problem described above and is based on the finding that the number of adipocytes can be controlled by electrical stimulation with a current at an appropriate frequency (20 kHz to 200 kHz) using a vector potential generator. Specifically, the present disclosure includes the following embodiments.
[1] A method for controlling the number of adipocytes in a tissue of a living body, including: imparting electrical stimulation to the tissue using a vector potential generator, wherein the electrical stimulation can increase or reduce the number of adipocytes by controlling a frequency of an alternating current applied to the vector potential generator.
[2] The method of [1], wherein the vector potential generator further has a cylindrical portion that includes a basic wire rod made up of a core wire with an insulating coating and an outer wire wound around the core wire with no gaps with the core wire serving as a winding axis and that is formed by winding the basic wire rod in a loop shape, one end of the core wire is electrically connected to one end of the outer wire, another end of the core wire is connected to one end of an external circuit, another end of the outer wire is connected to another end of the external circuit, and an alternating current is generated in the external circuit to impart electrical stimulation to a tissue placed in the cylindrical portion.
[3] The method of [1], wherein the vector potential generator further includes: a plurality of basic wire rods each made up of a core wire with an insulating coating and an outer wire wound around the core wire with no gaps with the core wire serving as a winding axis; and an external circuit that conducts an alternating current to the plurality of basic wire rods, and the plurality of basic wire rods are arrayed in a linear or curved array direction.
[4] The method of [1] or [2], wherein the tissue is a tissue subjected to limitation of motion, and the number of adipocytes is increased by controlling the frequency of the alternating current to lower than 50 kHz.
[5] The method of [4], wherein the frequency of the alternating current ranges from 1 to 30 kHz.
[6] The method of [1] or [2], wherein the tissue is an adipose tissue with obesity, and the number of adipocytes is reduced by controlling the frequency of the alternating current to 50 kHz or higher.
[7] The method of [6], wherein the frequency of the alternating current ranges from 100 to 300 KHz.
[8] The method of [2], wherein the alternating current is applied to the external circuit so that an electric field strength in the cylindrical portion ranges from 0.17 to 0.27 V/m.
[9] The method of [2], wherein the alternating current is applied to the external circuit so that an electric field strength in the cylindrical portion is 0.22 V/m.

### Advantageous Effect of Invention

The present invention enables the number of adipocytes to be increased or reduced by controlling the frequency of an alternating current applied to a vector potential generator.

### BRIEF DESCRIPTION OF THE DRAWINGS

FIG. 1 is a schematic view for explaining a basic wire rod that constitutes a vector potential generator used in a method of the present disclosure.

FIG. 2 is a schematic view for explaining a vector potential generator to which the basic wire rod shown in FIG. 1 is applied.

FIG. 3 is a schematic view of an experimental vector potential generator used in an example.

FIG. 4 is a diagram showing an arrangement of a basic wire rod in a vector potential generator of a modification of the present disclosure.

FIG. 5 is a diagram showing an application example of the vector potential generator of the modification of the present disclosure.

FIG. 6 is a diagram showing the number of adipocytes in a posterior portion of a rat's articular capsule measured in Example 1.

### EXPLANATION OF REFERENCE SIGNS

1 VP apparatus
5 tissue of a living body or a part thereof
8 external circuit
9 AC power supply
10,10a,10b,10c basic wire rod
20 cylindrical portion
21 core wire
22 outer wire

### DETAILED DESCRIPTION

Next, respective embodiments of the present disclosure will be described with reference to the drawings. Note that the respective embodiments described below are not intended to limit the invention as set forth in the accompanying claims and that all of the elements described in the embodiments and combinations thereof are not necessarily essential to solutions proposed by the present invention.

The present disclosure relates to a method for controlling the number of adipocytes in a tissue of a living body using a vector potential generator (hereinafter, referred to as a “VP apparatus”). Hereinafter, constituent elements in each embodiment will be described in order.

### <VP Apparatus>

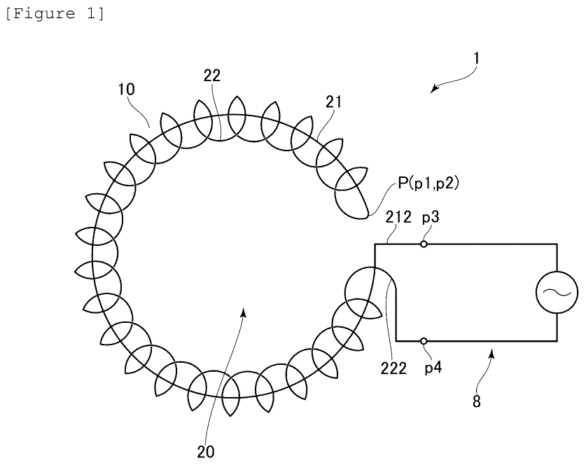

As can be seen in an outline thereof shown in FIG. 1, for example, a VP apparatus 1 used in the method of the present disclosure further has a cylindrical portion 20 that includes a basic wire rod 10 made up of a core wire 21 with an insulating coating and an outer wire 22 wound around the core wire 21 with no gaps with the core wire 21 serving as a winding axis and that is formed by winding the basic wire rod 10 in a loop shape, one end of the core wire 21 is electrically connected to one end of the outer wire 22, another end of the core wire 21 is connected to one end of an external circuit 8, and another end of the outer wire 22 is connected to another end of the external circuit 8.

The basic wire rod 10 is constituted of the core wire 21 and the outer wire 22 wound around the core wire 21 in a spiral shape. The core wire 21 and the outer wire 22 are separate conducting wires and one end parts p1 and p2 of the respective wires are connected at a point P. In addition, an end part p3 on a different side from the end part p1 of the core wire 21 and an end part p4 on a different side from the end part p2 of the outer wire 22 are, for example, end parts of a first lead-out wire 212 and a second lead-out wire 222 to be connected to the external circuit 8. The external circuit 8 is a circuit for sending electrical signals (for example, currents) input to the core wire 21 and the outer wire 22 and the external circuit 8 functions as a power supply device that supplies current. The VP apparatus 1 forms an electric field inside the cylindrical portion 20 constructed by winding the basic wire rod 10. In addition, the core wire 21 and the outer wire 22 are not limited to being separate conducting wires and can also be made of a single conducting wire that is folded back at the point P.

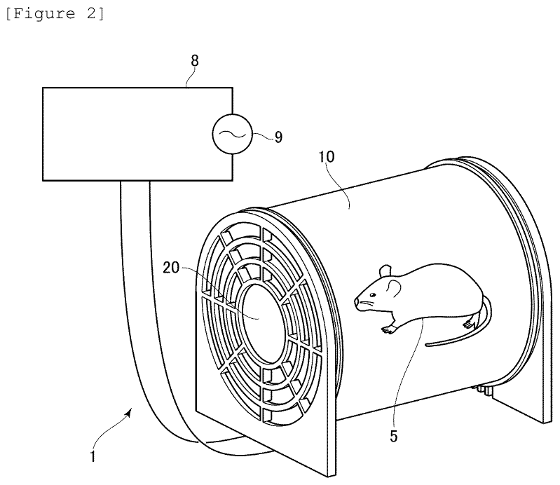

FIG. 2 is a schematic view for explaining the VP apparatus 1 to which the basic wire rod 10 shown in FIG. 1 is applied. The basic wire rod 10 is wound in a loop shape around the VP apparatus 1 by one or more turns and the cylindrical portion 20 for holding the living body or a tissue 5 thereof (a laboratory animal in the drawing) is formed inside the basic wire rod 10. At this point, in the cylindrical portion 20, turns of the basic wire rod 10 are preferably arrayed in a mutually gapless manner on an outer circumferential surface of adjacent turns of the basic wire rod 10. By holding the living body or the tissue thereof inside the cylindrical portion 20 and applying a current of a predetermined frequency to the basic wire rod 10 from an AC power supply 9 connected to the external circuit 8, an electric field is generated in an axial direction of the cylindrical portion 20 without contact and without generating a magnetic field inside the cylindrical portion 20. In addition, current flows inside the living body or the tissue 5 thereof held at a position within the electric field from a strong location to a weak location of the electric field.

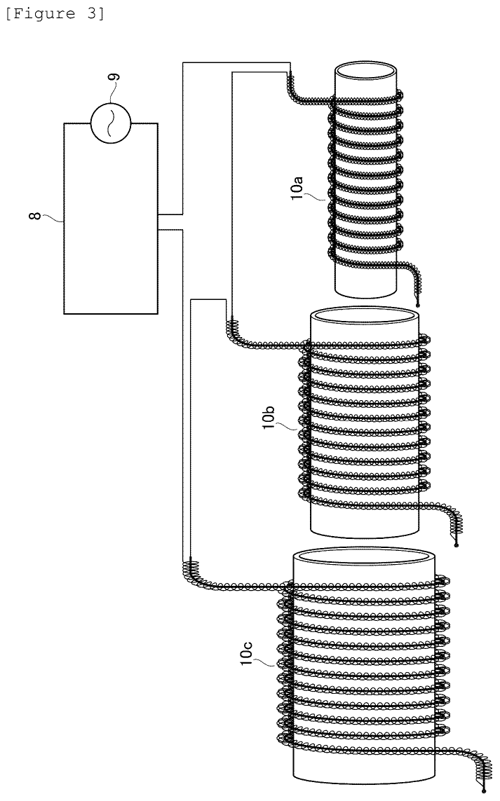

FIG. 3 is a schematic view of a VP apparatus used in another embodiment (an example to be described later). In the present embodiment, the basic wire rod 10 constituting the cylindrical portion 20 is made up of basic wire rods 10a, 10b, and 10c in a three-layer structure. Each basic wire rod has the core wire 21 and the outer wire 22 and is connected at one end of the basic wire rod 10. At another end, for example, the outer wire 22 of the basic wire rod 10a and the core wire 21 of the basic wire rod 10b are connected. In a similar manner, the basic wire rod 10b and the basic wire rod 10c are connected, and a current of a predetermined frequency is applied to a return wire of the basic wire rod 10a and the outer wire 22 of the basic wire rod 10c from the AC power supply 9.

Here, a voltage (electric field strength) created in the cylindrical portion 20 can be calculated based on a differential value of the current applied to the basic wire rods 10a, 10b, and 10c. Basically, the formula can be determined by a winding density, a coil diameter, and the like of the basic wire rod as described in detail in Patent Literature 2. As an example, the voltage created in the cylindrical portion 20 can be obtained based on the following equation (12) described in Patent Literature 2. Note that the entire contents described in Patent Literature 2 are incorporated herein by reference.

$$\begin{matrix}[ Expression 1 ] & \\ {V}_{2} = {μ}_{0} n {N}_{1} S ω ( \sqrt{{a}^{2} + {L}^{2}} - a ) {I}_{m} cos ( ω t ) & ( 12 )\end{matrix}$$

Here, V₂ denotes a voltage at which an electric field E due to a vector potential is accumulated, μ₀ denotes a permeability of vacuum, n denotes the number of turns of the outer wire per unit length of the core wire, Ni denotes the number of turns of the basic wire rod per unit length, S denotes a cross-sectional area of the basic wire rod, a denotes an inner radius of the cylindrical portion, L denotes the length of the basic wire rods 10a, 10b and 10c, Iₘ denotes an amplitude of the current, ω denotes a frequency, and t denotes time. Therefore, the voltage created in the cylindrical portion 20 can be controlled to a desired value by controlling the structure of the electrical stimulator such as a length, a diameter, the number of turns, and the like of a coil constituted of the basic wire rod wound in a loop shape and a frequency and an amplitude value of the current applied from the AC power supply 9.

Furthermore, the external circuit 8 can provide not only one cylindrical portion 20 but a plurality of cylindrical portions 20 attached to a plurality of tissues with similar or different currents simultaneously. In addition, downsizing of the external circuit 8 enables a battery-driven module or apparatus to be realized and further improves portability.

In addition, the external circuit 8 is desirably equipped with a control unit that controls parameters such as a magnitude, time, and a frequency of the current supplied to the core wire 21 and the outer wire 22 of the cylindrical portion 20. Furthermore, the control unit is capable of simultaneously controlling a plurality of cylindrical portions 20 and is preferably equipped with a function of changing the current described above and parameters such as a frequency based on data fed from other sensors such as a body temperature sensor or a bioelectric current sensor.

### <Modification of VP Generator>

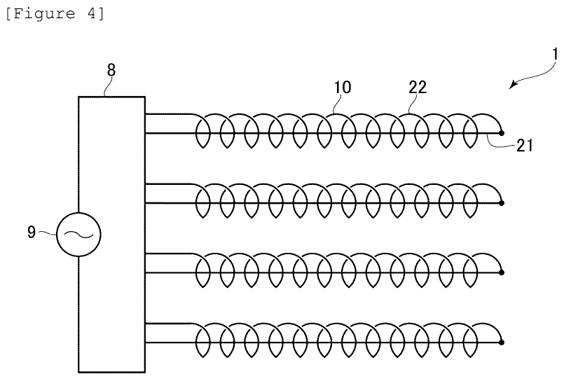

FIG. 4 is a diagram showing an arrangement of the basic wire rod 10 in the VP apparatus 1 of a modification of the present disclosure. In the modification, the VP apparatus 1 includes a plurality of basic wire rods 10. Each basic wire rod 10 in the modification is a plurality of solenoid coils having the core wire 21 with a linear shape and extending along the core wire 21. The plurality of basic wire rods 10 are arrayed in a linear array direction. In other words, the outer shape of the VP apparatus 1 is an abbreviated flat plate shape. The external circuit 8 conducts a current to the plurality of basic wire rods 10. The plurality of basic wire rods 10 may be electrically connected in series or in parallel. Alternatively, a plurality of external circuits 8 may conduct a current to each of the plurality of basic wire rods 10. In this case, the plurality of external circuits 8 conduct an alternating current to each of the plurality of basic wire rods 10 so that the alternating currents conducted to the plurality of basic wire rods 10 are synchronized. Furthermore, the core wire 21 that is a coil axis of the basic wire rod 10 may be constituted of a ferromagnetic member. The ferromagnetic member has a shape extending along the coil axis of the basic wire rod 10 with a shape of a solenoid coil and is formed of a ferromagnetic material. The ferromagnetic member is made of conductive material such as permalloy, and one end of the outer wire 22 and one end of the ferromagnetic member are electrically connected to each other to create a current path. In addition, the external circuit 8 applies a voltage to another end of the outer wire 22 and another end of the core wire 21 made of the ferromagnetic member to conduct a current to the basic wire rod 10.

In this manner, providing the basic wire rod 10 in plurality increases strength of a vector potential applied to an application object.

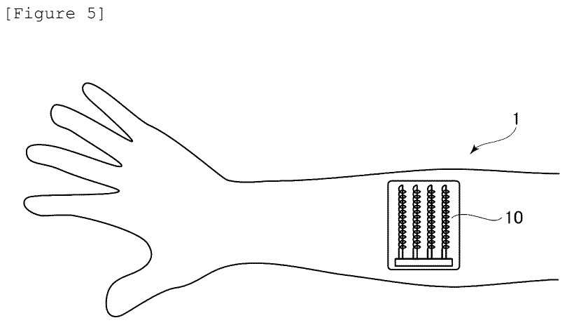

FIG. 5 is a diagram showing one example of the application of the VP apparatus 1 of the modification described above. For example, as shown in FIG. 5, the plurality of basic wire rods 10 are arranged on a sheet mounted in contact with or not in contact with the skin. By mounting the VP apparatus 1 on any tissue of the living body, a vector potential is applied to the tissue. While the basic wire rods 10 are arranged in a vertical direction with respect to a human elbow joint in FIG. 5, the basic wire rods 10 may be arranged in a longitudinal direction of the elbow joint (a longitudinal direction of an upper arm). In addition, while the basic wire rods 10 are arranged on a surface of the sheet in FIG. 5, the basic wire rods 10 may be built into a bag-like sheet.

### <Method for Controlling Number of Adipocytes>

The method for controlling the number of adipocytes of the present disclosure includes a step of applying electrical stimulation to a tissue of a living body using the VP apparatus described above. In this case, a living body refers to anything living including animals such as mammals including but not limited to primates (for example, humans), cows, sheep, goats, horses, dogs, cats, rabbits, rats, and mice, animals such as birds including but not limited to chickens, ducks, and turkeys, and animals such as fish including but not limited to eels, salmons, and horse mackerels. In a preferred embodiment, the subject is a human. In one embodiment, the electrical stimulation is provided by holding the tissue of the living body or a part of the living body inside a cylindrical portion of the VP apparatus described above and generating an alternating current in an external circuit for a predetermined time. Here, while the term “hold” means maintaining a position of the living body or a part thereof by fixing the living body or a part thereof in the cylindrical portion with a jig or the like, for example, maintaining the position of the living body or a part thereof in the cylindrical portion by fitting the living body or a part thereof into a concave surface or a concave curved surface and maintaining the position of the living body or a part thereof in the cylindrical portion by placing the living body or a part thereof on a flat surface also fall within the term “hold” as used herein. In a preferred embodiment, a flat loading stand or the like may be provided inside the cylindrical portion. In addition, an insulation material that does not conduct a current is preferable as a material of the loading stand. Rubber or a resin material such as polyethylene or polyvinyl chloride is more preferable. In addition, materials such as ceramics can also be used from the perspective of heat resistance.

In another embodiment, the VP apparatus of the modification described above with a sheet shape can be pasted to a living body or a part thereof and an electrical stimulation can be imparted by applying an alternating current from a power supply apparatus.

An electrical stimulation in the present disclosure enables the number of adipocytes to be increased or reduced by controlling a frequency of an alternating current applied to the VP apparatus. The term “controlling a frequency” refers to adjusting the frequency of a current applied to the VP apparatus to a specific range for obtaining a desired operational advantage and details thereof will be described later. The current may be a continuous alternating current or a pulse-like alternating current. In addition, as a preferred embodiment, a voltage pulse may be combined with a frequency as a parameter to be adjusted. Here, the term “combination of a voltage pulse” refers to any combination of one or more of (1) waveforms with different periods, (2) waveforms with different shapes (for example, a triangular wave, a sine wave, a square wave, and the like), and (3) waveforms with different duty ratios at different periods.

Furthermore, controlling the structure of the VP apparatus and current applied to the VP apparatus enables an electric field strength generated in the cylindrical portion to be controlled. The electric field strength can be appropriately adjusted according to a region or symptoms of a tissue to be an object and, although not limited, the electric field strength preferably ranges from approximately 0.1 to 1 V/m, more preferably, the electric field strength inside the cylindrical portion ranges from 0.17 to 0.27 V/m, and even more preferably the electric field strength is approximately 0.22 V/m. In this case, the strength of the electrical stimulation applied to the tissue held in the cylindrical portion can be estimated as, for example, a current value flowing in through the living body from the electric field strength applied to the electrical stimulator and an impedance of the tissue held in the cylindrical portion.

In some examples, a predetermined time during which the electrical stimulation is applied is the time during which the electrical stimulator of the present embodiment is activated to control the number of adipocytes. For example, the predetermined time is at least 30 minutes, 60 minutes, or 90 minutes per day, and the electrical stimulator is operated once, twice, or three times per day, continuously or discontinuously every day, or preferably at least five days a week for one to three weeks or more. This example of an operating time is not restrictive. Other therapies including additional exercise therapy and drug administration may be included.

In one embodiment of the present disclosure, the term “controlling the number of adipocytes” means increasing or maintaining (suppressing reduction of) the number of adipocytes. Typically, a method for increasing the number of adipocytes in a tissue of a living body is provided. Here, the tissue of the living body to be an object is preferably the tissue of the living body subjected to limitation of motion or a part thereof. The term “limitation of motion” refers to a restriction of the body's motor functions due to muscle tension, muscle weakness, or a decreased range of joint motion caused by diseases or trauma to the bone, muscle, or joint systems, diseases of the central nervous system, or the like. In addition, the method of the present embodiment includes imparting an electrical stimulation to the tissue subjected to limitation of motion using the VP apparatus to which an alternating current controlled to a specific frequency is conducted. The specific frequency is, for example, lower than 50 kHz, preferably 40 kHz or lower, more preferably 30 kHz or lower, and even more preferably 20 kHz or lower. Although a lower limit of the specific frequency is not particularly limited, for example, the lower limit is 0.1 kHz or higher, preferably 1 kHz or higher, and more preferably 2 kHz or higher. Typically, a range of 1 to 30 kHz is preferable.

Examples of a specific disease include “arthrogryposis (joint contracture)”. Joint contractures are often caused by immobilization or bed rest. Contractures may occur not only during local joint immobilization such as cast immobilization in treatment of soft tissue trauma or fractures, but also in neurological diseases and muscular diseases that require long-term treatment and care in the course of their illness. The changes in each tissue that cause contractures are thought to rarely exist independently of each other and a condition of restricted movement of a normal joint is generally broadly understood as a pathological concept of contracture. In the knee joint, there is abundant adipose tissue under the knee cap and its flexibility is believed to contribute to joint motion. It has been reported that limitation of joint motion (immobilization) results in atrophy and loss of infrapatellar adipose tissue. Therefore, by increasing the number of adipocytes by the method of the present embodiment, joint contractures can be prevented, treated, or improved.

Objects for treatment of joint contractures include, but are not limited to, contractures of the human shoulder joint, elbow joint, wrist joint, thumb joint, hip joint, and knee joint. It can also be used for similar purposes in non-human living bodies including companion animals such as dogs and cats and in the veterinary field including horses and, particularly, racehorses. For example, using the method of the present embodiment on a tissue subjected to limitation of motion during recuperation of an injured racehorse can shorten the recovery period from injury. In addition, in the production of livestock meat, the method of the present embodiment can be used on muscle tissue to increase the percentage of intramuscular fat to produce high quality beef and pork.

In another embodiment, the term “controlling the number of adipocytes” means reducing or maintaining (suppressing increase of) the number of adipocytes. Typically, a method for reducing the number of adipocytes in a tissue of a living body is provided. Here, the tissue of the living body to be an object is adipose tissue with obesity. A location of the adipose tissue is not particularly limited and may be subcutaneous fat or visceral fat. In addition, the method of the present embodiment includes imparting an electrical stimulation to the adipose tissue with obesity using the VP apparatus to which an alternating current controlled to a specific frequency is conducted. The specific frequency is, for example, 50 kHz or higher, preferably 80 kHz or higher, more preferably 100 kHz or higher, and even more preferably 200 kHz or higher. Although an upper limit of the specific frequency is not particularly limited, for example, the upper limit is 1000 kHz or lower, preferably 500 kHz or lower, and more preferably 300 kHz or lower. Typically, a range of 100 to 300 kHz is preferable.

Examples of specific diseases or application objects include overweight, obesity, metabolic disturbances, hypertension, lipid-related disorders, anorexia, and type II diabetes. The term “obese” refers particularly to subjects whose adipose tissue weight and body mass exceed the currently accepted norm. In some embodiments, subjects whose BMI exceeds the currently accepted norm are obese. In the case of human subjects, the current standard for both males and females accepted as “normal” is a BMI ranging from 20 to 24.9 kg/m². In the embodiments, a subject who is obese has a BMI of 30 kg/m² or higher. In some embodiments, a subject who is obese has a BMI of 40 kg/m² or higher. In another embodiment, a subject is obese if he/she weighs more than 120% of normal weight for his/her age and height. Normal body weight varies among species and individuals based on height, build, skeletal structure, and sex. The term “overweight” refers to moderately excess fat in a subject. In some embodiments, in the case of human subjects, a subject who is overweight has a BMI of 25 kg/m² or higher.

In yet other embodiments, exercise therapy, hyperthermic treatment, acupuncture, moxibustion, drug therapy, or the like is preferably combined with the method of the present disclosure to improve obesity. Drug therapy for obesity includes methods for promoting energy expenditure by increasing lipolysis in adipose tissue and methods for suppressing energy intake by inhibiting absorption of lipids and carbohydrates from the gastrointestinal tract and suppressing food intake. In addition, while these combination therapies may be able to inhibit adipocyte hypertrophy but not reduce their number, the method of the present disclosure can reduce the number of adipocytes and can be considered a permanently effective therapy to improve obesity.

While the present invention will be described below in greater detail by citing examples, it is to be understood that the present invention is not restricted in any way by the following examples.

### EXAMPLES

### <Experimental Apparatus>

A schematic view of a vector potential generator (hereinafter, referred to as a “VP apparatus”) used in the following examples is shown in FIG. 3. As shown in FIG. 3, three basic wire rods (VP wire rods) 10a, 10b, and 10c are wound around cylindrical portions. The three basic wire rods have the same length of 225 mm, the diameters of the cylindrical portions are different sizes of 130 mm, 170 mm and 210 mm, the number of turns is 97 T, the winding density of the winding wire is 950 T/m, and the basic wire rods are finally assembled concentrically. In addition, since the three basic wire rods that make up the VP apparatus are in series in the circuit, the basic wire rods actually correspond to a three-layer wound VP apparatus. The length of the apparatus is approximately 30 cm, and applying a 10.8 App sine wave to a VP coil results in an electric field strength of approximately 0.22 V/m in the longitudinal direction and a voltage of approximately 67 mV at both ends of the cylindrical portions. In addition, the operating frequency is 20 kHz. In the following examples, three different VP apparatuses were used, having different operating frequencies of 2 kHz or 200 kHz, respectively, while keeping the same electric field strength (approximately 0.22 V/m) inside the cylindrical portion by changing the number of turns of the basic wire rods, layer structure, and the magnitude of the applied current of the VP apparatuses.

### First Example

In the present example, using rats kept in a cage with an articular capsule fixed (hereafter referred to as “motion-limited reared rats”), the number of adipocytes in a posterior portion of the articular capsule was measured when the rats were subjected to stimulation by energization by an AC power supply of different frequencies using the VP apparatus described above.

### Experimental Method and Materials

Six male Wistar rats were used and randomly classified as follows.
CO group: Normal reared rat group.
IM group: Motion-limited reared rat group.
2 KHz group: Group of motion-limited reared rats irradiated with VP of 2 KHZ.
20 KHz group: Group of motion-limited reared rats irradiated with VP of 20 KHZ.
200 KHz group: Group of motion-limited reared rats irradiated with VP of 200 KHz

The VP-irradiated groups were energized for 30 minutes/day, 5 days/week, and 3 weeks under anesthesia at the respective frequencies. In this case, a voltage of approximately 67 mV is generated at both ends of the VP apparatus, and assuming that the rat held inside has an impedance of 500Ω, a current of 0.13 mA is estimated to flow. All of the groups were euthanized at the end of the 3-week experimental period and the tibiae were removed for histological observation.

### <Creation of Non-Decalcified Resin-Embedded Polished Specimens and Measurement of Number of Adipocytes>

After the end of the stimulation-by-energization experiment, the rats were euthanized by carbon dioxide aspiration, their skins were peeled, soft tissues were removed, and the tibiae were removed. The proximal tibia was sagittally fractured with a hand motor (Labo Force, manufactured by YOSHIDA DENTAL TRADE DISTRIBUTION CO., LTD.) fitted with a diamond disc (Meisinger, manufactured by GC Corp.) and promptly immersed in a fixative solution overnight. After washing the specimens in water, the specimens were dehydrated by an alcohol series. After permeabilization with acetone, the specimens were encapsulated in Rigolac resin and thermally polymerized in a thermostatic chamber (DY300, manufactured by Yamato Scientific Co., Ltd.). Blocks were trimmed with a band saw (K-100, manufactured by HOZAN CO., LTD.) and further rough-polished with a model trimmer (manufactured by YOSHIDA DENTAL TRADE DISTRIBUTION CO., LTD.). Surfaces were polished to a thickness of approximately 150 μm using three levels of grinding stones (coarse stone, medium stone, and finishing stone), and then carefully polished with a special film to remove surface scratches. After acid-etching the polished surface with 0.1 M hydrochloric acid, the surface was stained with a warmed 1% toluidine blue solution. The polished specimens were photographed with an optical microscope (BX53-33-FL-2, manufactured by Olympus Corporation) with a photographing apparatus (DP73-SET-B, manufactured by Olympus Corporation), and the number of adipocytes was measured visually.

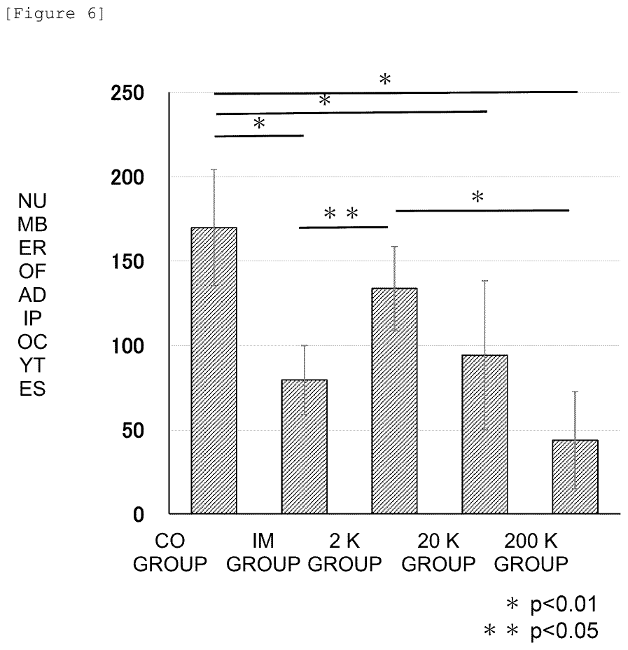

A result of the measurement is shown in Table 1 below and in FIG. 6.

TABLE 1

| | |
|---|---|
| Experimental | Number of adipocytes |

| group | Mean value | Standard deviation |
|---|---|---|
| CO group | 169.7 | 34.5 |
| IM group | 79.3 | 20.3 |
| 2 kHz group | 133.5 | 25.0 |
| 20 kHz group | 94.0 | 44.0 |
| 200 kHz group | 43.7 | 28.9 |

These results indicate that the number of adipocytes can be controlled by applying a displacement current to adipocytes in a non-contact manner using the VP apparatus. Specifically, in rats subjected to limitation of motion, the number of adipocytes significantly increased in the 2 kHz group electrically stimulated at a frequency of 2 kHz as compared to the group not subjected to electrical stimulation (IM group) (**p<0.05, see FIG. 6). On the other hand, the number of adipocytes decreased as the frequency increased from 2 kHz to 20 kHz and then to 200 kHz. The number of adipocytes in the 200 kHz group was further reduced than in the rats subjected to limitation of motion (IM group). These results suggest that adipocytes decrease as the frequency of the current applied to the VP apparatus increases, and that dieting and weight control can be achieved by reducing adipocytes even in situations where exercise is not possible.

### INDUSTRIAL APPLICABILITY

The method of controlling the number of adipocytes in the present disclosure is useful for promoting recovery from injury or joint contracture by increasing the number of adipocytes or for preventing or treating diseases caused by overweight or obesity by decreasing the number of adipocytes in a tissue of a living body.

1-9. (canceled)

10. A method for controlling the number of adipocytes in a tissue of a living body, comprising:
imparting electrical stimulation to the tissue using a vector potential generator, wherein the electrical stimulation can increase or reduce the number of adipocytes by controlling a frequency of an alternating current applied to the vector potential generator.

11. The method of claim 10, wherein the vector potential generator further has a cylindrical portion that includes a basic wire rod made up of a core wire with an insulating coating and an outer wire wound around the core wire with no gaps with the core wire serving as a winding axis and that is formed by winding the basic wire rod in a loop shape, one end of the core wire is electrically connected to one end of the outer wire, another end of the core wire is connected to one end of an external circuit, another end of the outer wire is connected to another end of the external circuit, and an alternating current is generated in the external circuit to impart electrical stimulation to the tissue placed in the cylindrical portion.

12. The method of claim 10, wherein the vector potential generator further includes: a plurality of basic wire rods each made up of a core wire with an insulating coating and an outer wire wound around the core wire with no gaps with the core wire serving as a winding axis; and
an external circuit that conducts an alternating current to the plurality of basic wire rods, and
the plurality of basic wire rods are arrayed in a linear or curved array direction.

13. The method of claim 10, wherein the tissue is a tissue subjected to limitation of motion, and the number of adipocytes is increased by controlling the frequency of the alternating current to lower than 50 KHz.

14. The method of claim 11, wherein the tissue is a tissue subjected to limitation of motion, and the number of adipocytes is increased by controlling the frequency of the alternating current to lower than 50 KHz.

15. The method of claim 13, wherein the frequency of the alternating current ranges from 1 to 30 KHz.

16. The method of claim 14, wherein the frequency of the alternating current ranges from 1 to 30 KHz.

17. The method of claim 10, wherein the tissue is an adipose tissue with obesity, and the number of adipocytes is reduced by controlling the frequency of the alternating current to 50 kHz or higher.

18. The method of claim 11, wherein the tissue is an adipose tissue with obesity, and the number of adipocytes is reduced by controlling the frequency of the alternating current to 50 kHz or higher.

19. The method of claim 17, wherein the frequency of the alternating current ranges from 100 to 300 KHz.

20. The method of claim 18, wherein the frequency of the alternating current ranges from 100 to 300 KHz.

21. The method of claim 11, wherein the alternating current is applied to the external circuit so that an electric field strength in the cylindrical portion ranges from 0.17 to 0.27 V/m.

22. The method of claim 11, wherein the alternating current is applied to the external circuit so that an electric field strength in the cylindrical portion is 0.22 V/m.
